(19) United States
(12) Patent Application Publication
(10) Pub. No.: US 2004/0156804 A1
(43) Pub. Date: Aug. 12, 2004

(54) Mascara comprising a polymer dispersed in a liquid fatty phase

(75) Inventors: Valerie de la Poterie, Le Chatelet En Brie (FR); Bertrand Piot, Paris (FR)

Correspondence Address: FINNEGAN, HENDERSON, FARABOW, GARRETT & DUNNER, LLP, 1300 I STREET, NW, WASHINGTON, DC, 20005, US

(73) Assignee: L'Oreal S.A.

(21) Appl. No.: 10/773,258

(22) Filed: Feb. 9, 2004

Related U.S. Application Data
(63) Continuation of application No. 09/598,637, filed on Jun. 21, 2000, now abandoned.

(30) Foreign Application Priority Data
Jun. 21, 1999 (FR) 99 08056

(51) Int. Cl. A61K 7/06; A61K 7/11

(52) U.S. Cl. 424/70.11; 424/70.12; 424/70.16; 424/70.17

(57) ABSTRACT

A composition for coating keratin fibers, in particular, eyelashes, including a dispersion of surface-stabilized polymer particles in a liquid fatty phase, the composition comprising at least 2% by weight, relative to the total weight of the composition, of the polymer particles and the composition having a viscosity, measured at 25° C., at a shear rate of 200 s⁻¹, ranging from 2 Pa·s to 17 Pa·s; a process for coating keratin fibers, which includes applying the composition to the keratin fibers; and a composition making it possible to obtain a coating for keratin fibers which withstands rubbing.

Th present invention relates to a composition for coating keratin fibers, comprising polymer particles dispersed in a liquid fatty phase. The invention also relates to the use of this composition for making up keratin fibers, as well as to a process for making up these fibers. The make-up composition and process according to the invention are intended more particularly for keratin fibers that are substantially longilinear, such as the eyelashes, the eyebrows and the hair of human beings, including false eyelashes and wigs. The composition can be a make-up composition, a make-up base, a composition to be applied to a make-up, also known as a topcoat, or a cosmetic treatment composition for keratin fibers. The invention more especially relates to a mascara.

Compositions for coating eyelashes, known as mascaras, generally comprise, in a known manner, at least one wax and a film-forming polymer to deposit a colored film on the eyelashes and to coat them, as described, for example, in International patent application WO-A-95/15741. A mascara composition comprising a small amount of polymer dispersed in isoparaffin is also known from European patent application EP-A-749 746.

However, these compositions do not make it possible to obtain a film that withstands rubbing, for example with the fingers or fabrics (handkerchiefs, towels) and the film disintegrates partially by crumbling or by spreading. Crumbling of the film gives rise to a substantial loss in the intensity of the color of the make-up, thus obliging the consumer to repeat the application of the make-up product. Spreading the film forms an aureole around the made-up zone, which is highly unesthetic. Water, in particular, atmospheric moisture during wet weather, tears and perspiration accentuate these drawbacks, as does the sebum of skin having a tendency towards being greasy. The film of make-up no longer has good overall staying power over time.

The aim of the present invention is to propose a composition preferably without the above drawbacks, preferably leading, when applied to keratin fibers, to the formation of a film having good staying power over time and withstanding rubbing, in particular, with the fingers or fabrics, under dry and/or wet conditions, and/or withstanding sebum and/or perspiration.

The inventors have discovered that such a composition can be obtained by using an effective amount of a polymer dispersed in a liquid fatty phase.

Specifically, a subject of the invention is a composition for coating keratin fibers, comprising, in a physiologically acceptable medium, a dispersion of surface-stabilized polymer particles in a liquid fatty phase, characterized in that it comprises at least 2% by weight, relative to the total weight of the composition, of the said polymer particles and in that the composition has a viscosity, measured at 25° C., at a shear rate of 200 s⁻¹, ranging from 2 Pa·s to 17 Pa·s.

A subject of the invention is also a process for coating keratin fibers, in particular, the eyelashes, which comprises applying a composition as described above to the keratin fibers.

A subject of the invention is also the use of a composition as defined above to obtain a film deposited on keratin fibers that withstands rubbing.

The composition according to the invention has a viscosity ranging from 2 Pa·s to 17 Pa·s (20 to 170 poises) and preferably from 5 Pa·s to 13 Pa·s (50 to 130 poises), to allow easy application of the composition to the keratin fibers, in particular, the eyelashes.

The viscosity is measured at 25° C. using a Rheomatt 180 viscometer sold by the company Rheo, at a shear rate of 200 s⁻¹, the measurement being carried out after rotating for 10 minutes (after which time stabilization of the viscosity and of the viscosity and of the spin speed of the spindle are observed).

The viscosity measurement is carried out using spindle No. 2 when the viscosity ranges from 20 centipoises to 7.32 poises (0.02 to 0.732 Pa—s); with spindle No. 3 for a viscosity ranging from 1.8 poises to 40.2 poises (0.18 to 4.02 Pa—s); with spindle No. 4 for a viscosity ranging from 80 poises to 1220 poises (8 to 122 Pa—s).

The composition according to the invention comprises a stable dispersion of generally spherical particles of at least one polymer, in a physiologically acceptable liquid fatty phase. These dispersions can be, in particular, in the form of nanoparticles of polymers as a stable dispersion in the said fatty phase. The nanoparticles preferably hav a size ranging from 5 to 600 nm, given that beyond about 600 nm, the particle dispersions become much less stable, and more preferably the size ranges from 50 nm to 250 nm.

The polymer used in the present invention can be of any nature. It is thus possible to use a radical-mediated polymer, a polycondensate or even a polymer of natural origin and mixtures thereof. The polymer can be chosen by a person skilled in the art on the basis of its properties.

One of the advantages of the polymer dispersion in the composition of the invention is the possibility of varying the glass transition temperature (Tg) of the polymer or of the polymer system (polymer plus additive of plasticizer type), and of thus passing from a soft polymer to a more or less hard polymer, allowing adjustment of the mechanical properties of the film obtained with the composition of the invention.

The polymers usable in the composition of the invention preferably have a weight-average molecular weight ranging from about 2000 to 10,000,000. The polymer can preferably have a glass transition temperature ranging from −100° C. to 300° C. and, better still, from −10° C. to 50° C.

It is possible to use film-forming polymers, preferably having a low glass transition temperature, less than or equal to the temperature of the skin, and, in particular, less than or equal to about 40° C. The expression “film-forming polymer” means a polymer capable by itself, or in the presence of a plasticizer, of forming an isolable film. A dispersion is thus obtained which can form a film when it is applied to a support, this not being the case when dispersions of mineral pigments according to the prior art are used.

When the polymer has a glass transition temperature too high for the desired application, it can be combined with a plasticizer to lower the glass transition temperature of the mixture used. The plasticizer can be chosen from the plasticizers usually used in the field of application, and, in particular, from compounds that may be solvents for the polymer.

Examples of film-forming polymers include radical-mediated, acrylic or vinyl homopolymers or copolymers, preferably having a Tg of less than or equal to about 40° C., and especially ranging from −10° C. to 30° C., and mixtures thereof.

Examples of non-film-forming polymers include radical-mediated, vinyl or acrylic homopolymers or copolymers preferably having a Tg greater than about 40° C., and especially ranging from 45° C. to 100° C., and mixtures thereof. The non-film-forming polymer makes it possible, in combination with the liquid fatty phase, to form a continuous and homogeneous deposit on the eyelashes.

The expression “a radical-mediated polymer” means a polymer obtained by polymerization of monomers containing unsaturation, in particular, ethylenic unsaturation, each monomer being capable of homopolymerizing (unlike polycondensates). The radical-mediated polymers can be, in particular, vinyl polymers or copolymers, in particular, acrylic polymers.

The vinyl polymers can result from the polymerization of monomers containing ethylenic unsaturation having at least one acid group and/or esters of these acidic monomers and/or amides of these acids.

Monomers bearing an acid group that can be used are a, β-ethylenic unsaturated carboxylic acids, such as acrylic acid, methacrylic acid, crotonic acid, maleic acid or itaconic acid. (Meth)acrylic acid and crotonic acid are preferably used, and, more preferably, (meth)acrylic acid.

The acid monomer esters are advantageously chosen from (meth)acrylic acid esters (also known as (meth)acrylates), especially alkyl(meth)acrylates, in particular, of a C₁-C₂₀ alkyl, preferably of a C₁-C₈ alkyl, aryl(meth)acrylates, in particular, of a C₆-C₁₀ aryl, and hydroxyalkyl(meth)acrylates, in particular, of a C₂-C₆ hydroxyalkyl.

Examples of alkyl(meth)acrylates include methyl(meth)acrylate, ethyl (meth)acrylate, butyl(meth)acrylate, isobutyl(meth)acrylate, 2-ethylhexyl(meth) acrylate and lauryl(meth)acrylate.

Examples of hydroxyalkyl(meth)acrylates include hydroxyethyl(meth)acrylate and 2-hydroxypropyl(meth)acrylate.

Examples of aryl(meth)acrylates include benzyl acrylate and phenyl acrylate.

The (meth)acrylic acid esters that are particularly preferred are the alkyl(meth)acrylates.

Copolymers of (meth)acrylic acid and of an alkyl(meth)acrylate, in particular, a C₁-C₄ alkyl, are preferably used as radical-mediated polymer. More preferably, methyl acrylates optionally copolymerized with acrylic acid can be used.

Examples of amides of the acid monomers include (meth)acrylamides, and especially N-alkyl(meth)acrylamides, in particular, a C₂-C₁₂ alkyl, such as N-ethylacrylamide, N-t-butylacrylamide, N-octylacrylamide; N-di(C₁-C₄)alkyl(meth)acrylamides.

The vinyl polymers can also result from the polymerization of monomers containing ethylenic unsaturation having at least one amine group, in free form or in partially or totally neutralized form, or, alternatively, in partially or totally quatemized form. Such monomers can be, for example, dimethylaminoethyl(meth)acrylate, dimethylaminoethyl-methacrylamide, vinylamine, vinylpyridine or diallyldimethylammonium chloride.

The vinyl polymers can also result from the homopolymerization or copolymerization of at least one monomer chosen from vinyl esters and styrene monomers. In particular, these monomers can be polymerized with acid monomers and/or esters thereof and/or amides thereof, such as those mentioned above.

Examples of vinyl esters include vinyl acetate, vinyl propionate, vinyl neodecanoate, vinyl pivalate, vinyl benzoate and vinyl t-butylbenzoate.

Examples of styrene monomers include styrene and alpha-methylstyrene.

The list of monomers given is not limiting and it is possible to use any monomer known to those skilled in the art falling within the categories of acrylic and vinyl monomers (including monomers modified with a silicone chain).

Other vinyl monomers which may also be mentioned are:

N-vinylpyrrolidone; vinylcaprolactam; vinyl-N-(C₁-C₆)alkylpyrroles; vinyloxazoles, vinylthiazoles; vinylpyrimidines; vinylimidazoles;

olefins, such as ethylene, propylene, butylene, isoprene or butadiene.

The vinyl polymer can be crosslinked using a difunctional monomer, in particular, comprising at least two ethylenic unsaturations, such as ethylene glycol dimethacrylate or diallyl phthalate.

In a non-limiting manner, the polymers of the invention can be chosen from the following polymers or copolymers: polyurethanes, polyurethane-acrylics, polyureas, polyurea-polyurethanes, polyester-polyurethanes, polyether-polyurethanes, polyesters, polyesteramides, polyesters containing a fatty chain, alkyds; acrylic-silicone copolymers; polyacrylamides; silicone polymers and fluoro polymers, and mixtures thereof.

With such a dispersion of polymer particles, it is possible to calibrate the size of the polymer particles as desired, and to modify their size “polydispersity” during the synthesis. It is thus possible to obtain particles of very small size, invisible to the naked eye when in the composition and when applied to the skin or the lips. This would not be possible with pigments in particulate form, since their constitution does not allow the average size of the particles to be modified.

The dispersed polymer is used in an amount effective to obtain a film deposited on keratin fibers that withstands rubbing, possibly in the presence of water and/or sebum and/or perspiration.

In practice, the polymer dispersed in the liquid fatty phase can be present in a content ranging from 2% to 50% by weight, relative to the total weight of the composition, preferably from 4% to 40% by weight and, better still, from 5% to 30% by weight.

The expression “liquid fatty phase” means any non-aqueous medium liquid at room temperature (25° C.) and atmospheric pressure. This fatty phase can contain a volatile liquid fatty phase and/or a non-volatile liquid fatty phase containing one or more liquid oils, respectively.

The expression “volatile fatty phase” means any non-aqueous medium capable of evaporating from the skin in less than one hour. This volatile phase, in particular, comprises oils with a vapour pressure, at room temperature and atmospheric pressure, ranging from 10⁻³ to 300 mmHg (0.13 Pa to 40,000 Pa).

The liquid fatty phase in which the polymer is dispersed can comprise any physiologically acceptable and, in particular, cosmetically acceptable oil, chosen, in particular, from oils of mineral, animal, plant or synthetic origin, carbon-based, hydrocarbon-based, fluoro and/or silicone oils, alone or as a mixture provided that they form a mixture, preferably homogeneous and stable, and provided that they are compatible with the envisaged use.

The total liquid fatty phase of the composition can preferably represent from 5% to 98% by weight, relative to the total weight of the composition, and more preferably from 20 to 85% by weight. The non-volatile part can preferably represent from 0 to 80% (more in particular, from 0.1 to 80%) of the total weight of the composition and, better still, from 1 to 50%.

Examples of liquid fatty phase usable in the invention include hydrocarbon-based oils, such as liquid paraffin, liquid petroleum jelly, mink oil, turtle oil, soybean oil, perhydrosqualene, sweet almond oil, beauty-leaf oil, palm oil, grape seed oil, sesame oil, corn oil, parleam oil, arara oil, rapeseed oil, sunflower oil, cotton oil, apricot oil, castor oil, avocado oil, jojoba oil, olive oil or cereal germ oil; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl triisostearate or diglyceryl triisostearate; higher fatty acids, such as myristic acid, palmitic acid, stearic acid, behenic acid, oleic acid, linoleic acid, linolenic acid or isostearic acid; higher fatty alcohols, such as cetanol, stearyl alcohol, oleyl alcohol, linoleyl alcohol, linolenyl alcohol, isostearyl alcohol or octyldodecanol; silicone oils such as polydimethylsiloxanes (PDMSs), optionally phenylated, such as phenyltrimethicones, or optionally substituted with aliphatic and/or aromatic groups, optionally fluorinated, or optionally substituted with functional groups, such as hydroxyl, thiol and/or amine groups; polysiloxanes modified with fatty acids, fatty alcohols or polyoxyalkylenes, fluorosilicones, perfluoro oils.

Advantageously, one or more oils that are volatile at room temperature can be used. These volatile oils are favorable for obtaining a film with total “transfer-resistance” properties, i.e., a film totally resistant to rubbing. After evaporation of these oils, a non-sticky, flexible, film-forming deposit is obtained. These volatile oils also make it easier to apply the composition to keratin fibers, such as the eyelashes.

These volatile oils can be hydrocarbon-based oils or silicone oils optionally comprising alkyl or alkoxy groups at the end of the silicone chain or pendant on the chain.

Examples of volatile silicone oils that can be used in the invention include linear or cyclic silicones containing from 2 to 7 silicon atoms, these silicones optionally comprising alkyl or alkoxy groups containing from 1 to 10 carbon atoms. Particular examples include octamethylcyclotetrasiloxane, decamethylcyclopentasiloxane, hexadecyl-methylcyclohexasiloxane, heptamethylhexyltrisiloxane and heptamethyloctyltrisiloxane.

Volatile hydrocarbon-based oils that may be mentioned are C₈-C₁₆ isoparaffins, such as ‘Isopar’s and ‘Permetyl’s and, in particular, isododecane.

These volatile oils can be present in the composition in a content ranging from 5 to 97.99% relative to the total weight of the composition, and preferably from 30 to 75%.

In one particular embodiment of the invention, the liquid fatty phase is chosen from:

non-aqueous liquid compounds having a global solubility parameter according to the Hansen solubility space of less than 17 (MPa)^{1/2},

or monoalcohols having an overall solubility parameter according to the Hansen solubility space of less than or equal to 20 (MPa)^{1/2},

or mixtures thereof.

The global solubility parameter δ global according to the Hansen solubility space is defined in the article “Solubility parameter values” by Eric A. Grulke in the book “Polymer Handbook” 3rd Edition, Chapter VII, pages 519-559, the disclosure of which is incorporated herein by reference, by the relationship:

δ=(d_{D}²+d_{P}²+d_{H}²)^{1/2}

in which

d_{D} characterizes the London dispersion forces arising from the formation of dipoles induced during molecular impacts,

d_{P} characterizes the Debye interaction forces between permanent dipoles,

d_{H} characterizes the forces of specific interactions, such as hydrogen bonding, acid/base, donor/acceptor, etc. The definition of solvents in the three-dimensional solubility space according to Hansen is described in the article by C. M. Hansen: “The three-dimensional solubility parameters”, J. Paint Technol. 39, 105 (1967), the disclosure of which is incorporated herein by reference.

Examples of liquid fatty phases having a global solubility parameter according to the Hansen solubility space of less than or equal to 17 (Mpa)^{1/2} include plant oils formed from fatty acid esters of polyols, in particular, triglycerides, such as sunflower oil, sesame oil or rapeseed oil, or esters derived from acids or alcohols containing a long chain, i.e., a chain containing from 6 to 20 carbon atoms, in particular, the esters of formula RCOOR′ in which R represents a higher fatty acid residue containing from 7 to 19 carbon atoms and R′ represents a hydrocarbon-based chain containing from 3 to 20 carbon atoms, such as palmitates, adipates and benzoates, in particular, diisopropyl adipate. Other examples include hydrocarbons and, in particular, liquid paraffin, liquid petroleum jelly or hydrogenated polyisobutylene, isododecane or ‘Isopar’s, and volatile isoparaffins. Mention may also be made of silicone oils, such as polydimethylsiloxanes and polymethylphenylsiloxanes, optionally substituted with aliphatic and/or aromatic groups, optionally fluorinated, or optionally substituted with functional groups, such as hydroxyl, thiol and/or amine groups, and volatile silicone oils, in particular, volatile cyclic silicone oils. Mention may also be made of solvents, alone or as a mixture, chosen from (i) linear, branched or cyclic esters containing more than 6 carbon atoms, (ii) ethers containing more than 6 carbon atoms, (iii) ketones containing more than 6 carbon atoms. The expression “monoalcohols having a global solubility parameter according to the Hansen solubility space of less than or equal to 20 (MPa)^{1/2}” a means aliphatic fatty alcohols containing at least 6 carbon atoms, the hydrocarbon-based chain comprising no substitution groups. Examples of monoalcohols according to the invention include oleyl alcohol, decanol, dodecanol, octadecanol and linoleyl alcohol.

Non-aqueous media which can also be used are those described in French patent application FR-A-2 710 646 from L.V.M.H., the disclosure of which is incorporated herein by reference.

The choice of the non-aqueous medium is made by a person skilled in the art on the basis of the nature of the monomers constituting the polymer and/or the nature of the stabilizer, as indicated below.

The polymer dispersion can be manufactured as described in European patent application EP-A-749 747, the disclosure of which is incorporated herein by reference. The polymerization can be carried out by dispersion, i.e., by precipitating the polymer while it is forming, with protection of the particles formed with a stabilizer.

A mixture is thus prepared comprising the initial monomers as well as a radical initiator. This mixture is dissolved in a solvent, referred to in the rest of the present description as the “synthesis solvent.” When the fatty phase is a non-volatile oil, the polymerization can be carried out in an apolar organic solvent (synthesis solvent) followed by addition of the non-volatile oil (which should be miscible with the synthesis solvent) and selective distillation of the synthesis solvent.

A synthesis solvent is thus chosen such that the initial monomers and the radical initiator are soluble therein, and the polymer particles obtained are insoluble therein, in order for them to precipitate therein as they are formed. In particular, the synthesis solvent can be chosen from alkanes, such as heptane, isododecane or cyclohexane.

When the fatty phase chosen is a volatile oil, the polymerization can be carried out directly in the oil which thus also acts as synthesis solvent. The monomers should also be soluble therein, as should the radical initiator, and the polymer obtained should be insoluble therein.

The monomers are preferably present in the synthesis solvent, before polymerization, in a proportion of 5-20% by weight of the reaction mixture. The total amount of the monomers can be present in the solvent before the start of the reaction, or some of the monomers can be added gradually as the polymerization reaction proceeds.

The radical initiator can be, in particular, azobis(isobutyronitrile) or tert-butylperoxy-2-ethyl hexanoate.

The polymer particles are surface-stabilized, gradually as the polymerization proceeds, by a stabilizer that can be a block polymer, a grafted polymer, and/or a random polymer, alone or as a mixture. The stabilization can be carried out by any known means, and, in particular, by direct addition of the block polymer, grafted polymer and/or random polymer during the polymerization.

The stabilizer is preferably also present in the mixture before polymerization. However, it is also possible to add it continuously, in particular, when the monomers are also added continuously.

2-30% by weight of stabilizer can preferably be used relative to the initial monomer mixture, and preferably 5-20% by weight.

When a grafted and/or block polymer is used as stabilizer, the synthesis solvent is chosen such that at least some of the grafts or blocks of the stabilizing polymer are soluble in the solvent, the rest of the grafts or blocks being insoluble therein. The stabilizing polymer used during the polymerization should be soluble, or dispersible, in the synthesis solvent. Furthermore, a stabilizer whose insoluble blocks or grafts have a certain affinity for the polymer formed during the polymerization is preferably chosen.

Examples of grafted polymers include silicone polymers grafted with a hydrocarbon-based chain and hydrocarbon-based polymers grafted with a silicone chain.

Grafted copolymers having, for example, an insoluble skeleton of polyacrylic type with soluble grafts of poly(12-hydroxystearic acid) type are also suitable for use.

Grafted-block or block copolymers comprising at least one block of polyorganosiloxane type and at least one block of a radical-mediated polymer can also be used, such as grafted copolymers of acrylic/silicone type that can be used, in particular, when the non-aqueous medium is a silicone medium.

The stabilizer can also be chosen from grafted-block or block copolymers comprising at least one block of polyorganosiloxane type and at least one block of polyether type. The polyorganopolysiloxane block can be, in particular, a polydimethylsiloxane or a poly(C₂-C₁₈)alkylmethylsiloxane; the polyether block can be a poly(C₂-C₁₈)alkylene, in particular, polyoxyethylene and/or polyoxypropylene. In particular, dimethicone copolyols or (C₂-C₁₈)alkylmethicone copolyols can be used. It is possible, for example, to use the dimethicone copolyol sold under the name “Dow Coming 3225C” by the company Dow Corning, or the lauryl methicone copolyol sold under the name Dow Corning Q2-5200″ by the company Dow Corning.

Grafted-block or block copolymers that can be used are copolymers comprising at least one block resulting from the polymerization of at least one ethylenic monomer, containing one or more optionally conjugated ethylenic bonds, such as ethylene, butadiene or isoprene, and of at least one block of a styrene polymer. When the ethylenic monomer comprises several optionally conjugated ethylenic bonds, the residual ethylenic unsaturations after the polymerization are generally hydrogenated. Thus, in a known manner, the polymerization of isoprene leads, after hydrogenation, to the formation of ethylene-propylene blocks, and the polymerization of butadiene leads, after hydrogenation, to the formation of ethylene-butylene blocks. Among these block copolymers which may be mentioned are copolymers of “diblock” or “triblock” type, such as polystyrene/polyisoprene or polystyrene/polybutadiene, such as those sold under the name ‘Luvitol HSB’ by BASF, of the polystyrene/copoly(ethylene-propylene) type, such as those sold under the name ‘Kraton’ by Shell Chemical Co. or, alternatively, of the polystyrene/copoly(ethylene-butylene) type.

Examples of grafted-block or block copolymers comprising at least one block resulting from the polymerization of at least one ethylenic monomer, such as ethylene or isobutylene, and of at least one block of an acrylic polymer, such as methyl methacrylate include poly(methyl methacrylate)/polyisobutylene diblock or triblock copolymers or grafted copolymers with a poly(methyl methacrylate) skeleton and with polyisobutylene grafts.

Examples of grafted-block or block copolymers comprising at least one block resulting from the polymerization of at least one ethylenic monomer and of at least one block of a polyether, such as a C₂-C₁₈ polyoxyalkylene, in particular, polyoxyethylene and/or polyoxypropylene include polyoxyethylene/polyoxybutadiene or polyoxyethylene/polyisobutylene diblock or triblock copolymers.

Copolymers of C₁-C₄ alkyl(meth)acrylates and of C₈-C₃₀ alkyl(meth)acrylates can also be used. Mention may also be made of the stearyl methacrylate/methyl methacrylate copolymer.

When a random polymer is used as stabilizer, it is chosen such that it has a sufficient amount of groups to make it soluble in the synthesis solvent envisaged.

When the synthesis solvent is apolar, the stabilizer preferably chosen is a polymer that covers the particles as completely as possible, several stabilizing-polymer chains then becoming adsorbed on a polymer particle obtained by polymerization.

In this case, the stabilizer preferably used is either a grafted polymer or a block polymer, so as to have better interfacial activity, because the blocks or grafts that are insoluble in the synthesis solvent provide more voluminous coverage at the surface of the particles.

When the liquid synthesis solvent comprises at least one silicone oil, the stabilizer is preferably chosen from grafted-block or block copolymers comprising at least one block of polyorganosiloxane type and at least one block of a radical-mediated polymer or of a polyether or a polyester, such as polyoxy(C₂-C₁₈)alkylene blocks and, in particular, polyoxypropylenated and/or polyoxyethylenated blocks.

When the liquid fatty phase does not comprise a silicone oil, the stabilizer is preferably chosen from:

(a) grafted-block or block copolymers comprising at least one block of polyorganosiloxane type and at least one block of a radical-mediated polymer or of a polyether or a polyester,

(b) copolymers of C₁-C₄ alkyl acrylates or methacrylates and of C₈-C₃₀ alkyl acrylates or methacrylates,

(c) grafted-block or block copolymers comprising at least one block resulting from the polymerization of at least one ethylenic monomer containing conjugated ethylenic bonds, and at least on block of a vinyl or acrylic polymer or of a polyether or a polyester, of mixtures thereof.

Preferably, diblock polymers are used as stabilizer.

Depending on the application, dispersions of film-forming or non-film-forming polymers in volatile or non-volatile oils may be chosen.

The composition according to the invention can also contain at least one wax that can be chosen from waxes of animal origin, waxes of plant origin or waxes of synthetic origin.

The waxes useful in the composition according to the invention generally have a melting point between 40 and 110° C. The wax can also have a needle penetration ranging from 1 to 217. The needle penetration of the waxes is determined according to French standard NF T 60-123 or US standard ASTM D 1321, at a temperature of 25° C. According to these standards, the needle penetration is a measurement of the depth, expressed in tenths of a millimeter, to which a standardized needle, weighing 2.5 g mounted in a mobile assembly weighing 97.5 g and placed on the wax to be tested, for 5 seconds, penetrates into the wax.

Examples of waxes of animal origin include beeswaxes, lanolin waxes and Chinese insects waxes.

Examples of waxes of plant origin include rice waxes, carnauba wax, candelilla wax, ouricury wax, cork fiber waxes, sugar cane waxes, Japan waxes, sumach wax and cotton wax.

Examples of waxes of mineral origin include paraffins, microcrystalline waxes, montan waxes and ozokerites.

Particular examples of usable waxes of synthetic origin include polyolefin waxes and, in particular, polyethylene waxes, the waxes obtained by Fischer-Tropsch synthesis, waxy copolymers and esters thereof, and silicone waxes.

It is also possible to use hydrogenated oils of animal or plant origin that still satisfy the two physical characteristics mentioned above.

Among these oils, mention may be made of hydrogenated oils obtained by catalytic hydrogenation of fatty substances composed of linear or non-linear C₈-C₃₂ fatty chains, hydrogenated sunflower oil, hydrogenated castor oil, hydrogenated coconut oil, hydrogenated jojoba oil, hydrogenated lanolin and hydrogenated palm oils.

The waxes that can be used according to the present invention are preferably solid and rigid at a temperature below 50° C.

The composition according to the invention can comprise from 0% to 30%, in particular, from 0.1% to 30%, by weight of wax, relative to the total weight of the composition, preferably from 1% to 25% by weight.

Preferably, the composition according to the invention can comprise:

at least one wax having a needle penetration ranging from 1 to 7.5 (referred to as wax I), in particular, in a content ranging from 0.1% to 20% by weight relative to the total weight of the composition, and

at least one wax having a needle penetration of greater than 7.5 and less than or equal to 217 (referred to as wax II), in particular, in a content ranging from 0.1% to 10% by weight relative to the total weight of the composition.

The composition can also contain a polymer dissolved in the liquid fatty phase, also referred to as a liposoluble polymer.

Particular examples of lipophilic polymers include the copolymers resulting from the copolymerization of at least one vinyl ester and of at least one other monomer that may be an olefin, an alkyl vinyl ether or an allylic or methallylic ester, as described in French patent application FR-A-2 262 303, the disclosure of which is incorporated herein by reference.

Examples of liposoluble film-forming polymers usable within the invention include polyalkylenes and, in particular, copolymers of C₂-C₂₀ alkenes, such as polybutene, alkyl celluloses with a linear or branched, saturated or unsaturated C₁ to C₈ alkyl radical, such as ethyl cellulose and propyl cellulose, copolymers of vinylpyrrolidone (VP) and, in particular, copolymers of vinylpyrrolidone, and a C₂ to C₄₀ and, better still, C₃ to C₂₀ alkene. Examples of VP copolymers include the copolymer of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

The liposoluble film-forming polymer can be present in the composition in a content preferably ranging from 0.1% to 15% by weight relative to the total weight of the composition, and, better still, from 2% to 10% by weight.

The composition can also comprise at least one dyestuff, such as pulverulent compounds and/or liposoluble dyes, for example in a proportion of from 0.01 to 50% relative to the total weight of the composition. The pulverulent compounds can be chosen from the pigments and/or nacres usually used in cosmetic compositions. Advantageously, the pulverulent compounds represent from 0.1 to 25% relative to the total weight of the composition and, better still, from 1 to 20%.

The pigments may be white or colored, and mineral and/or organic. Among the mineral pigments that may be mentioned are titanium dioxide, which is optionally surface-treated, zirconium oxide or cerium oxide, as well as iron oxide or chromium oxide, manganese violet, ultramarine blue, chromium hydrate and ferric blue. Examples of organic pigments include carbon black, pigments of D & C type and lakes based on cochineal carmine, barium, strontium, calcium or aluminium.

The nacreous pigments can be chosen from white nacreous pigments, such as mica coated with titanium or with bismuth oxychloride, colored nacreous pigments, such as titanium mica with iron oxides, titanium mica with, in particular, ferric blue or chromium oxide, titanium mica with an organic pigment of the abovementioned type, as well as nacreous pigments based on bismuth oxychloride.

The composition can also comprise fillers that may be chosen from those well known to those skilled in the art and commonly used in cosmetic compositions. The fillers may be mineral or organic, and lamellar-or spherical. Mention may be made of talc, mica, silica, kaolin, Nylon powder (Orgasol from Atochem), poly-β-alanine powder and polyethylene powder, Teflon, lauroyllysine, starch, boron nitride, tetrafluoroethylene polymer powders, hollow microspheres such as Expancel (Nobel Industrie), Polytrap (Dow Corning) and silicone resin microbeads (Tospearls from Toshiba, for example), precipitated calcium carbonate, magnesium carbonate and hydrocarbonate, hydroxyapatite, hollow silica microspheres (Silica Beads from Maprecos), glass or ceramic microcapsules, metal soaps derived from organic carboxylic acids containing from 8 to 22 carbon atoms, preferably from 12 to 18 carbon atoms, for example zinc, magnesium or lithium stearate, zinc laurate or magnesium myristate.

The composition according to the invention can also comprise a thickener for the liquid fatty phase. The thickener can be chosen from organomodified clays, which are clays treated with compounds chosen, in particular, from quatemary amines and tertiary amines. Organomodified clays that may be mentioned are organomodified bentonites, such as those sold under the name “Bentone 34” by the company Rheox, and organomodified hectorites such as those sold under the names “Bentone 27” and “Bentone 38” by the company Rheox.

Treated silicas and liposoluble alkylated guar gums can also be used.

The thickener can be present in an amount to make it possible to adjust the viscosity to the desired value. It may be present, in particular, in a content ranging from 0.1% to 10% by weight relative to the total weight of the composition.

The composition can also contain any additive usually used in such compositions, such as fragrances, preserving agents, surfactants, plasticizers, sequestering agents, vitamins, proteins, ceramides, acidifying or basifying agents, and emollients.

Needless to say, a person skilled in the art will take care to select this or these optional additional compound(s), and/or the amount thereof, such that the advantageous properties of the composition according to the invention are not, or are not substantially, adversely affected by the addition envisaged.

According to one embodiment of the invention, the composition can be advantageously anhydrous, and can contain less than 10% of water relative to the total weight of the composition. In this case, it can be present in the form of an oily gel or a paste.

The present invention is further illustrated by the following examples which are designed to teach those of ordinary skill in the art how to practice the invention. The following examples are merely illustrative of the invention and should not be construed as limiting the invention as claimed.

The viscosity of each composition was measured at 25° C. using a Rheomatt 180 viscometer sold by the company Rheo, equipped with a spindle No. 3 or 4, at a shear rate of 200 s⁻¹, the measurement being made after spinning the spindle for 10 minutes.

### EXAMPLE 1 OF A POLYMER DISP RSION

A dispersion of a non-crosslinked copolymer of methyl acrylate and of acrylic acid in an 85/15 ratio, in isododecane, was prepared according to the method of Example 7 of European patent application EP-A-749 747, the disclosure of which is incorporated herein by reference. A dispersion of poly(methyl acrylate/acrylic acid) particles in isododecane that were surface-stabilized with a polystyrene/copoly(ethylene-propylene) diblock copolymer sold under the name Kraton G1701 (Shell), having a solids content of 22.6% by weight and an average particle size of 175 nm (polydispersity: 0.05) and a Tg of 20° C., was thus obtained. This copolymer could form a film.

### EXAMPLES 2 TO 5 OF MASCARA (COMPARATIVE)

Four compositions 2 to 5 below were prepared, composition 5 containing no bentonite; composition 2, containing only 1.13% solids of the polymer of the dispersion of Example 1, did not form part of the invention.

| Polymer dispersion of Example 1 | x | g |
|---|---|---|
| Paraffin | 2.2 | g |
| Carnauba wax | 4.5 | g |
| Beeswax | 8.3 | g |
| Vinyl acetate/allyl stearate copolymer | 2.2 | g |
| (65/35) (Mexomere PG | | |
| from Chimex) | | |
| Polyvinyl laurate (Mexomere | 0.75 | g |
| PP from Chimex) | | |
| Rice starch | 0.85 | g |
| Bentonite | 5.32 | g |
| Propylene carbonate | 1.74 | g |
| Pigments | 4.6 | g |
| Preserving agents | qs | |
| Isododecane | qs 100 | g |

For each composition, a film 300 μm thick (before drying) was spread on a glass plate and, after drying for 24 hours at 30° C., the film was then rubbed with a cotton bud soaked in water or in Bifacil make-up remover from Lancôme. The number of wipes with the cotton bud needed to damage the film is counted.

The results below were obtained:

| | Example | | | |
|---|---|---|---|---|
| | 2 (NWI) | 3 | 4 | 5 |
| X (solids)* | 5 (1.13) | 10 (2.23) | 30 (6.78) | 50 (11.3) |
| Viscosity in Pa · s | 5.9 (59) | 6.2 (62) | 13 (130) | 0.69 (6.9) |
| (in poises) | spindle 4 | spindle 4 | spindle 4 | spindle 3 |
| Number of wipes | | | | |
| Water | 8 | 12 | 20 | 72 |
| Bifacil | 13 | 15 | 42 | 70 |

NWI: Not within the invention
Solids*: Content in grams, expressed as solids of polymer particles of the dispersion of Example 1.

It was found that the compositions of Examples 3 to 5 according to the invention gave a film that fully withstood rubbing in the presence of water, whereas the composition of Example 2 not forming part of the invention led to a film which withstood rubbing less well.

### EXAMPLE 6 OF A POLYMER DISPERSION

A dispersion of non-crosslinked copolymer of methyl methacrylate and of acrylic acid in an 85/15 ratio, in isododecane, was prepared according to the method of Example 1 of European patent application EP-A-749 746, the disclosure of which is incorporated herein by reference, replacing the heptane with isododecane. A dispersion of poly(methyl methacrylate/acrylic acid) particles in isododecane which were surface-stabilized with a polystyrene/copoly(ethylene-propylene) diblock copolymer sold under the name Kraton G1701 (Shell), having a solids content of 25.5% by weight and an average particle size of 150 nm (polydispersity <0.1) and a Tg of 100° C., was thus obtained. This copolymer could not form a film.

### EXAMPLE 7 OF A MASCARA

A mascara having the composition described in Example 3 was prepared using 10 g of the polymer dispersion of Example 6, i.e., 2.55 g of solids. The composition had a viscosity, measured with spindle No. 4, of 7.8 Pa·s (78 poises). This composition was tested according to the test described in Examples 2 to 5. It was found that the film was damaged after wiping it 50 times with the cotton bud soaked in water, and after wiping it 40 times with the cotton bud soaked in Bifacil make-up remover. The composition of Example 7 thus gave a film which fully withstood rubbing.

The composition applied easily to the eyelashes and gave a make-up effect which withstood rubbing, in particular, with the fingers.

What is claimed is:

1. A composition for coating keratin fibers, comprising a dispersion of surface-stabilized polymer particles in a liquid fatty phase, wherein said polymer particles comprise at least 2% by weight relative to the total weight of the composition, and wherein said composition has a viscosity, measured at 25° C., at a shear rate of 200 s⁻¹, ranging from 2 Pa·s to 17 Pa·s.

2. A composition according to claim 1, wherein said composition is in a physiological acceptable medium.

3. A composition according to claim 1, wherein said viscosity ranges from 5 Pa·s to 13 Pa·s.

4. A composition according to claim 1, wherein said polymer is chosen from radical-mediated polymers, polycondensates and polymers of natural origin, and mixtures thereof.

5. A composition according to claim 1, wherein said polymer is chosen from polyurethanes, polyurethane-acrylics, polyureas, polyurea-polyurethanes, polyester-polyurethanes, polyether-polyurethanes, polyesters, polyesteramides, polyesters containing a fatty chain, alkyds; acrylic and vinyl polymers and acrylic and vinyl copolymers; silicone polymers and fluoro polymers, and mixtures thereof.

6. A composition according to claim 1, wherein said polymer is a film-forming polymer.

7. A composition according to claim 1, wherein said polymer particles are stabilized with a stabilizer chosen from block polymers, grafted polymers and random polymers, and mixtures thereof.

8. A composition according to claim 7, wherein said stabilizer is chosen from silicone polymers grafted with a hydrocarbon-based chain; hydrocarbon-based polymers grafted with a silicone chain; grafted-block and block copolymers comprising at least one polyorganosiloxane block and at least one block of a radical-mediated polymer; grafted-block and block copolymers comprising at least one polyorganosiloxane block and at least one polyether block; copolymers of C₁-C₄ alkyl(meth)acrylates and of C₈-30alkyl(meth)acrylates; grafted-block and block copolymers comprising at least one block resulting from the polymerization of ethylenic monomers comprising at least one optionally conjugated ethylenic bond and at least one styrene polymer block; grafted-block and block copolymers comprising at least one block resulting from the polymerization of ethylenic monomers and at least one acrylic polymer block; grafted-block and block copolymers comprising at least one block resulting from the polymerization of an ethylenic monomer and at least one polyether block.

9. A composition according to claim 7, wherein said stabilizer is chosen from a grafted-block and block polymers comprising at least one block resulting from the polymerization of ethylenic monomers comprising at least one optionally conjugated ethylenic bond and at least one styrene polymer block.

10. A composition according to claim 1, wherein said polymer particles are present in a solids content ranging from 2% to 50% by weight relative to the total weight of the composition.

11. A composition according to claim 10, wherein said polymer particles are present in a solids content ranging from 4% to 40% by weight relative to the total weight of the composition.

12. A composition according to claim 11, wherein said polymer particles are present in a solids content ranging from 5% to 30% by weight relative to the total weight of the composition.

13. A composition according to claim 1, wherein said liquid fatty phase comprises at least one oil chosen from mineral, animal, plant, synthetic origin and hydrocarbon-based oils, fluoro oils and silicone oils.

14. A composition according to claim 1, wherein said liquid fatty phase comprises at least one substance from liquid paraffin, liquid petroleum jelly, mink oil, turtle oil, soybean oil, perhydrosqualene, sweet almond oil, beauty-leaf oil, palm oil, parleam oil, grape seed oil, sesame oil, corn oil, rapeseed oil, sunflower oil, cotton oil, apricot oil, castor oil, avocado oil, jojoba oil, olive oil, cereal germ oil; esters of lanolic acid, of olelc acid, of laucric acid and of stearic acid; fatty esters; higher fatty acids; higher fatty alcohols; silicone oils; polysiloxanes modified with fatty acids, fatty alcohols other than said higher fatty alcohols, polyoxyalkylenes, fluorosilicones, perfluoro oils; and volatile oils differing from any of said oils recited above in this claim.

15. A composition according to claim 14, wherein said fatty esters are chosen from isopropyl myristate, isopropyl palmiate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, glyceryl trilsostearate and diglyceryl triisostearate.

16. A composition according to claim 14, wherein said higher fatty acids are chosen from myristic acid, palmitic acid, stearic acid, behenic acid, oleic acid, linoleic acid, linolenic acid and isostearic acid.

17. A composition according to claim 14, wherein said higher fatty alcohols are chosen from cetanol, stearyl alcohol, oleyl alcohol, linoleyl alcohol, linolenyl alcohol, isostearyl alcohol and octyldodecanol.

18. A composition according to claim 14, wherein said silicone oils are chosen from polydimethylsiloxanes which are optionally phenylated, optionally substituted with at least one aliphatic and aromatic group, and optionally substituted with functional groups.

19. A composition according to claim 18, wherein said polydimethylsiloxanes which are optionally phenylated are chosen from phenyltrimethicones.

20. A composition according to claim 18, wherein said functional groups are chosen from hydroxyl, thiol and amine groups.

21. A composition according to claim 14, wherein said volatile oils are chosen from octamethylcyclotetrasiloxane, decamethylcyclopentasiloxane, hexadecamethylcyclohexasiloxane, heptamethylhexyltrisiloxane, heptamethyloctyltrisiloxane and C₈-C₁₆ isoparaffins.

22. A composition according to claim 14, wherein said at least one substance is isododecane.

23. A composition according to claim 1, wherein said liquid fatty phase is chosen from:
non-aqueous liquid compounds having a global solubility parameter according to the Hansen solubility space of less than 17 (MPa)^{1/2},
monoalcohols having an overall solubility parameter according to the Hansen solubility space of less than or equal to 20 (MPa)^{1/2}, and
mixtures thereof.

24. A composition according to claim 1, wherein said fatty phase contains at least one oil, and wherein said at least one oil is volatile at room temperature.

25. A composition according to claim 1, wherein said composition further comprises at least one additive chosen from fragrances, preserving agents, surfactants, plasticizers, sequestering agents, vitamins, proteins, ceramides, acidifying or basifying agents, emollients, dyestuffs and fillers.

26. A composition according to claim 1, wherein said composition is anhydrous.

27. A composition for coating keratin fibers, comprising a dispersion of surface-stabilized polymer particles in a liquid fatty phase, wherein said polymer particles comprise at least 2% by weight relative to the total weight of the composition, and wherein said composition has a viscosity, measured at 25° C., at a shear rate of 200 s⁻¹, ranging from 2 Pa·s to 17 Pa·s, wherein said composition is a make-up composition, a make-up base, a composition to be applied to a make-up, or a cosmetic treatment composition for keratin fibers.

28. A method of coating keratin fibers, comprising applying to said keratin fibers, a composition which comprises a dispersion of surface-stabilized polymer particles in a liquid fatty phase, wherein said polymer particles comprise at least 2% by weight relative to the total weight of the composition, and wherein said composition has a viscosity, measured at 25° C., at a shear rate of 200 s⁻¹, ranging from 2 Pa·s to 17 Pa·s.

29. A method according to claim 28, wherein said keratin fibers are eyelashes.

30. A method of treating keratin fibers, comprising applying to said keratin fibers, a composition to obtain a film, wherein said composition comprises a dispersion of surface-stabilized polymer particles in a liquid fatty phase, wherein said polymer particles comprise at least 2% by weight relative to the total weight of the composition, and wherein said composition has a viscosity, measured at 25° C., at a shear rate of 200 s⁻¹, ranging from 2 Pa·s to 17 Pa·s.

31. A method according to claim 30, wherein said keratin fibers are eyelashes.
